(19) United States
(12) Patent Application Publication
(10) Pub. No.: US 2024/0058556 A1
(43) Pub. Date: Feb. 22, 2024

(54) DETECTING THE PRESENCE OF LIQUID IN A VIBRATING MEMBRANE NEBULIZER

(71) Applicant: VECTURA DELIVERY DEVICES LIMITED, Chippenham (GB)

(72) Inventor: Yannic WEISER, Cambridge (GB)

(21) Appl. No.: 18/268,079

(22) PCT Filed: Dec. 15, 2021
(86) PCT No.: PCT/EP21/85980

§ 371 Date: Jun. 16, 2023

(30) Foreign Application Priority Data
Dec. 16, 2020 (EP) 20214452.3

(51) Int. Cl. A61M 15/00; A61M 16/00

(52) U.S. Cl. CPC A61M 15/0085; A61M 15/0021; A61M 16/0003; A61M 2016/0024; A61M 2016/0027; A61M 2205/0294

(57) ABSTRACT

The present invention provides a breath-actuated inhalation device comprising: an aerosol generator comprising a vibrator and a membrane; and a reservoir for liquid to be aerosolized which is in fluid communication with the membrane. A method for operating the device is also provided. The vibrator is driven intermittently so that the aerosol generator has periods of aerosol generation during a patients inhalations and periods of little or no aerosol generation preceding and/or succeeding the inhalations Scans are performed in which an electrical parameter of the vibrator is measured as the membrane is vibrated at a plurality of frequencies. The spectrum obtained from a scan during an inhalation is compared with a spectrum obtained from a scan during the period preceding or succeeding that inhalation in order to determine whether liquid is present in the reservoir.

### TECHNICAL FIELD OF THE INVENTION

The present invention relates to a vibrating membrane nebulizer, and in particular to a method for detecting the presence of liquid in contact with the membrane.

### BACKGROUND TO THE INVENTION

Aerosols for medical inhalation therapy generally comprise an active ingredient dissolved or suspended in an aerosolisable liquid, often water. A homogeneous distribution of aerosol droplets with a droplet size of around 5 μm is required in order to reach deep into the lungs. Vibrating membrane nebulizers are one type of device for producing such aerosols. These devices have an aerosol generator which comprises a vibrator, such as piezoelectric element which is excited at ultrasonic frequencies in order to induce vibration; a membrane (sometimes called a mesh or aperture plate), which has a large number of holes which typically have a diameter of 1 μm to 10 μm; and a reservoir, which supplies the liquid drug formulation to the membrane.

Continuing to operate a vibrating membrane nebulizer after the liquid reservoir has been emptied can cause the membrane to crack or break. Therefore, it is important to be able to reliably detect whether or not liquid is present in the reservoir/in contact with the membrane (“empty detection”). When the liquid has been used up, the nebulizer can indicate the end of the treatment session to the patient and/or turn off the vibrator automatically.

One approach is simply to measure the amount of liquid in the reservoir with a suitable sensor. For example, US2006/0255174 discloses a nebulizer in which the amount of liquid in the reservoir is sensed by a piezoelectric sensor, an optical sensor, a conductivity sensor or a strain gauge. However this requires a dedicated sensor, which adds to the cost and complexity of the device; also, the sensor may need to be in contact with the liquid, which can present problems.

An alternative approach exploits the fact that the vibration characteristics of the aerosol generator (e.g. resonant frequency, power consumption etc.) are typically quite different when the membrane is in contact with liquid compared to when it is dry. For example, US2006/0102172 discloses a nebulizer which determines whether liquid is present or not by comparing the detected value of an electrical parameter (such as the current to the piezoelectric element) at a particular frequency with a stored value. U.S. Pat. No. 9,272,101 uses impedance measurements in a similar manner. Instead of using a single frequency, the electromechanical behaviour of the aerosol generator can be expressed as a spectrum, for example a graph of power or current consumption as a function of the vibration frequency. WO2014/062175 and WO2015/091356 disclose methods for comparing measured and stored spectra, using averages and various mathematical techniques, to improve the reliability of empty detection. A comparison is typically performed at regular intervals while the membrane is being vibrated during the treatment session, until it is identified that the liquid has all been used up.

However, inherent variations in the hardware components of the nebulizer (e.g. aerosol generator, driver electronics) and/or changes in these over the lifetime of the nebulizer, or due to changes in the external conditions, such as the ambient temperature, can affect the electro-mechanical behaviour. For example, methods which rely on comparisons with pre-set values can produce erroneous outcomes if the properties of the aerosol generator and/or driver electronics are differ from those on which the standard, stored values are based. Consequently, the vibrator could continue vibrating after the liquid has been used up, which could damage the membrane; or vibration could be stopped while liquid still remains to be aerosolized, so that the full dose is not delivered to the patient. Thus there remains a need for improved methods for reliably detecting the presence of liquid in the reservoir/or in contact with the membrane.

### BRIEF DESCRIPTION OF THE INVENTION

The present invention provides an improved method for determining when the liquid to be nebulized has been used up. The inventor recognized that transient effects can be observed in the spectra immediately after vibration starts or stops, and that these can be used to reliably detect the presence or absence of liquid in contact with the membrane. Accordingly, in a first aspect, the present invention provides a breath-actuated inhalation device comprising: an aerosol generator comprising a vibrator and a membrane; a reservoir for liquid to be aerosolized which is in fluid communication with the membrane; and a controller which provides a driver signal to drive the vibrator so that the membrane vibrates and generates an aerosol; wherein the controller is configured to:
drive the vibrator intermittently so that the aerosol generator repeatedly has periods of aerosol generation during a patient's inhalations and periods of little or no aerosol generation preceding and/or succeeding the inhalations;
perform scans in which the membrane is vibrated at a plurality of frequencies, and in which at least one electrical parameter of the vibrator is measured at the plurality of frequencies to provide a spectrum; wherein scans are performed during the inhalations and during the periods preceding or succeeding the inhalations;
compare a spectrum obtained during an inhalation with a spectrum obtained during the period preceding or succeeding that inhalation;
determine whether liquid is present in the reservoir on the basis of the comparison of the spectra; and
cease to drive the vibrator if the controller determines that no liquid is present.

Previous empty detection methods that measure the values of an electrical parameter while the membrane is being vibrated rely on changes in the electro-mechanical properties of the aerosol generator as the volume of liquid decreases over time. In contrast, the present invention relies on an effect which occurs when the membrane is vibrated intermittently, as in a breath-actuated nebulizer. The invention compares spectra obtained during periods of aerosol generation and periods of little or no aerosol generation to identify changes which arise from the formation or dissipation of a standing wave in the reservoir when vibration is started or stopped respectively. The invention is much less dependent on the properties of aerosol generator than the previous methods because it does not rely on a comparison with, for example, a stored spectrum; instead it detects a transient change that occurs only when a liquid is present. Consequently, the invention is more robust to variations in the hardware, changes in the hardware over the lifetime of the nebulizer and changes in external conditions.

The controller may be configured to perform a first scan before each inhalation or period of aerosol generation to obtain a first spectrum; to subsequently perform a second scan during each inhalation or period of aerosol generation to obtain a second spectrum; and to compare the first and second spectra. The first scan may be performed immediately before the start of the period of aerosol generation. The second scan may be performed at least 50 or 100 ms after the start of the period of aerosol generation. This provides sufficient time for the standing wave to form, so that it is detectable in the second scan. The second scan may be performed less than 1000 ms or 500 ms after the start of the period of aerosol generation. The time delay between the first and second scans determines the point at which the absence of liquid can be detected. A smaller delay results in earlier empty detection, so the vibration of the membrane can be stopped sooner.

The inhalation device is breath-actuated, i.e. the aerosol is not generated continuously, but only when the patient inhales. The device may comprise a channel having an air inlet opening and an aerosol outlet opening, and a pressure sensor which is pneumatically connected to the channel, and the controller may be configured to: receive a signal from the pressure sensor; sense an inhalation by a patient at the aerosol outlet opening on the basis of the signal from the pressure sensor; and initiate a period of aerosol generation in response to the inhalation. The controller may be configured to initiate a period of little or no aerosol generation at a pre-set time after the period of aerosol generation was initiated. Alternatively, the controller may be configured to sense when the patient ceases to inhale on the basis of the signal from the pressure sensor and to initiate a period of little or no aerosol generation in response to the cessation of inhalation. The method of the invention is particularly suitable for breath-actuated nebulizers, because there is no need to change to the manner in which the nebulizer operates since the aerosol is inherently generated intermittently.

The aerosol generator may comprise a support member on which the vibrator and/or the membrane are mounted. The vibrator may be an annular piezoelectric element. The support element may be a transducer in the form of a hollow tubular body having a flange at or close to a first end onto which the piezoelectric element is attached, and a second end into or onto which the membrane is mounted. The device may comprise a filling chamber located above, and in fluid communication with, the support member, so that the filling chamber and the hollow tubular body together form the reservoir. Alternatively, the support member may comprise an essentially planar annulus or disk, and the membrane and/or the vibrator may be mounted on the support member, for example on opposite sides.

The controller may be configured to determine a resonant frequency of the aerosol generator from the spectra, and to drive the vibrator at the resonant frequency, or at a frequency that is related to the resonant frequency, such as a fixed offset from the resonant frequency, during the periods of aerosol generation, other than the scans.

The plurality of frequencies may comprise from about 10 or 15 kHz below the resonant frequency to about 10 or 15 kHz above the resonant frequency, for example from about 75 kHz to about 100 kHz.

The controller may be configured to perform the comparison of the spectra by calculating an overlap function. The controller may further be configured to determine that no liquid is present in the reservoir if the value of the overlap function is above a threshold value. The controller may also be configured to cease driving the vibrator if the overlap function is above the threshold for a plurality of consecutive inhalations or periods of aerosol generation, such as three or five inhalations or periods of aerosol generation. The overlap function provides a robust comparison method.

In a second aspect, the invention provides a method of operating an inhalation device of the first aspect of the invention, and/or of operating a breath-actuated inhalation device comprising an aerosol generator comprising a vibrator and a membrane, and a reservoir for liquid to be aerosolized which is in fluid communication with the membrane, the methods comprising:
a) driving the vibrator intermittently so that the aerosol generator repeatedly has periods of aerosol generation during a patient's inhalations and periods of little or no aerosol generation preceding and/or succeeding the inhalations;
b) performing scans in which the membrane is vibrated at a plurality of frequencies, and in which at least one electrical parameter of the vibrator is measured at the plurality of frequencies to provide a spectrum; wherein scans are performed during the inhalations and periods of little or no aerosol generation;
c) comparing a spectrum obtained during an inhalation with a spectrum obtained during the period preceding or succeeding that inhalation; d) determining whether liquid is present in the reservoir on the basis of the comparison of the spectra; and
e) ceasing to drive the vibrator if it is determined in step d) that no liquid is present.

A first scan may be performed before each inhalation to obtain a first spectrum, a second scan may subsequently be performed during each inhalation to obtain a second spectrum, and the first and second spectra may be compared.

When the inhalation device comprises a channel having an air inlet opening and an aerosol outlet opening, and a pressure sensor which is pneumatically connected to the channel, the periods of aerosol generation may be initiated in response to inhalation by the patient on the basis of a signal from the pressure sensor. The periods of little or no aerosol generation may be initiated at a pre-set time after the period of aerosol generation was initiated. Alternatively, the periods of little or no aerosol generation may be initiated in response to the cessation of inhalation by the patient.

The spectra may be compared by calculating an overlap function. It may be determined that no liquid is present in the reservoir if the overlap function is above a threshold value. Driving the vibrator may cease if the overlap function is above the threshold value for a plurality of consecutive inhalations or periods of aerosol generation, such as three or five inhalations or periods of aerosol generation.

In a specific embodiment, the invention provides a breath-actuated inhalation device comprising: an aerosol generator comprising a vibrator and a membrane; a reservoir for liquid to be aerosolized which is in fluid communication with the membrane; and a controller which provides a driver signal to drive the vibrator so that the membrane vibrates and generates an aerosol in the channel; wherein the controller is configured to:
drive the vibrator intermittently so that the aerosol generator repeatedly has periods of aerosol generation during a patient's inhalations and periods of little or no aerosol generation preceding and/or succeeding the inhalations;
immediately before, or at the start of each inhalation or period of aerosol generation, perform a first scan in which the membrane is vibrated at a plurality of frequencies, and in which at least one electrical parameter of the vibrator is measured for each of the plurality of frequencies to provide a first spectrum;
subsequently during each inhalation or period of aerosol generation, perform a second scan in which the membrane is vibrated at the plurality of frequencies, and in which the at least one electrical parameter of the vibrator is measured for each of the plurality of frequencies to provide a second spectrum;
perform a comparison of the first and second spectra;
determine whether liquid is present in the reservoir on the basis of the comparison of the first and second spectra; and
cease to drive the vibrator if the controller determines that no liquid is present.

The invention also provides a method of operating a breath-actuated inhalation device according to this specific embodiment, the method comprising:
a) driving the vibrator intermittently so that the aerosol generator repeatedly has periods of aerosol generation during a patient's inhalations and periods of little or no aerosol generation preceding and/or succeeding the inhalations;
b) immediately before, or at the start of each inhalation or period of aerosol generation, performing a first scan in which the membrane is vibrated at a plurality of frequencies, and in which at least one electrical parameter of the vibrator is measured for each of the plurality of frequencies to provide a first spectrum;
c) subsequently during each inhalation or period of aerosol generation, performing a second scan in which the membrane is vibrated at the plurality of frequencies, and in which the at least one electrical parameter of the vibrator is measured for each of the plurality of frequencies to provide a second spectrum;
d) performing a comparison of the first and second spectra;
e) determining whether liquid is present in the reservoir on the basis of the comparison of the first and second spectra; and
f) ceasing to drive the vibrator if it is determined in step e) that no liquid is present.

### BRIEF DESCRIPTION OF THE FIGURES

The invention will now be further described with reference to the Figures, wherein:

FIG. 1 shows an expanded view of a vibrating membrane nebulizer.

FIG. 2 is a cross-sectional view through the aerosol generator for the nebulizer of FIG. 1.

FIG. 3 is a schematic diagram of the driver circuit for the aerosol generator.

FIG. 4 shows schematic drawings of the liquid surface in the reservoir before (FIG. 4A) and during (FIG. 4B) vibration of the membrane.

FIG. 5 shows spectra obtained without liquid in the reservoir.

FIG. 6 shows spectra obtained with liquid in the reservoir.

FIG. 7 shows a graph of the overlap function over the course of a representative treatment.

### DETAILED DESCRIPTION OF THE INVENTION

The term “period of aerosol generation” refers to a period of time in which the vibrator is mainly driven at the normal, intended frequency for generating an aerosol, which is typically at or near (e.g. within 2 kHz of) the resonant frequency. A period of aerosol generation may also include short periods of time in which one or more scans are performed. A period of aerosol generation may correspond to the typical length of a patient's inhalation, such as from 1 to 10 s, 2 to 6 s, or 3 to 5 s. The term “period of little or no aerosol generation” refers to the intervals between periods of aerosol generation in which the vibrator is mainly not driven. A period of little or no aerosol generation may also include short periods of time in which one or more scans are performed. Since most of the scan frequencies are quite far (e.g. more than 2 kHz) from the resonant frequency, little or no aerosol is generated during a scan. Consequently, little or no aerosol is generated in a period of little or no aerosol generation. A period of little or no aerosol generation may correspond to the typical time between a patient's inhalations, such as from 1 to 10 s, 2 to 6 s, or 3 to 5 s. Thus, intermittently driving the vibrator (at the normal driving frequency) results in alternate periods of aerosol generation and no aerosol generation.

The term “scan” refers to the process of sequentially vibrating the vibrator at a large number of different frequencies in stepwise increments across a defined range, and measuring the value of an electrical parameter at some or all of the frequencies. The term “spectrum” refers to a graph which is obtained by plotting the measured values of the electrical parameter as a function of frequency. The electrical parameter may be the current, voltage, power, impedance and/or the current/voltage phase shift. In particular, the electrical parameter may be the current consumption of the vibrator, or of a power converter which provides the power to the vibrator, or the voltage drop at the vibrator. These parameters can be measured by using one or more current and/or voltage sensors, in a direct or an indirect manner.

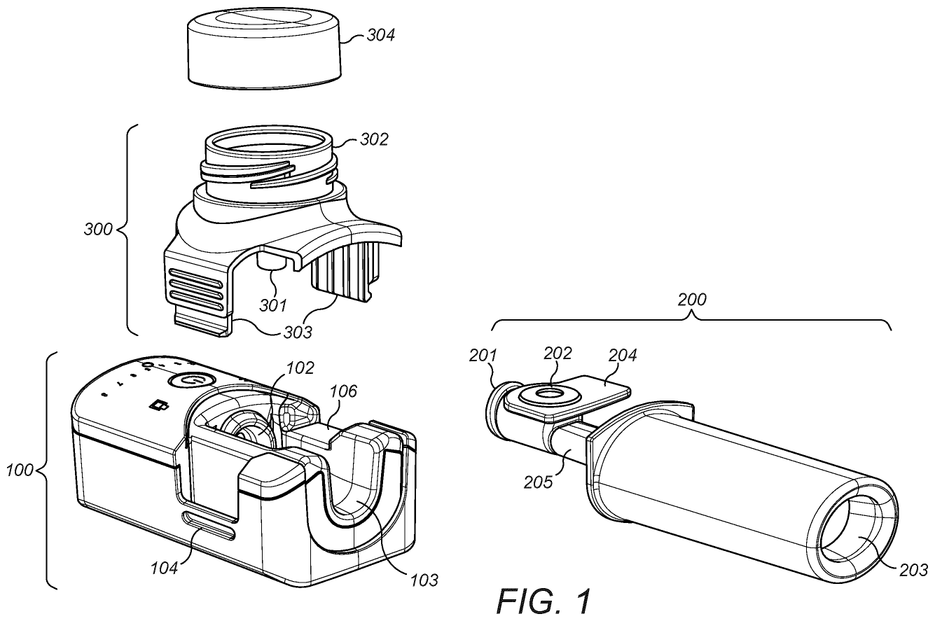

FIG. 1 shows an expanded view of a vibrating membrane nebulizer device, which is described in EP2724741 and WO2013/098334. The device comprises three parts: a base unit, a mouthpiece component, and an aerosol head. The base unit 100 has one or more air inlet opening(s) in its rear end (not visible in FIG. 1), an air outlet opening 102, a groove 103 for receiving the mouthpiece component 200, and one or more key lock members 104. A channel within the base unit (not visible in FIG. 1) connects the air inlet opening(s) to the air outlet opening 102. The mouthpiece component 200 has an air inlet opening 201 which is attachable to the air outlet opening 102 of the base unit 100, a lateral opening 202 for receiving an aerosol generator 301, and an aerosol outlet opening 203. A channel 205 extends from the air inlet opening 201 to the aerosol outlet opening 203. The mouthpiece 200 is insertable into the groove 103 of the base unit 100. The aerosol head 300 comprises the aerosol generator 301, a filling chamber 302 for the liquid drug formulation to be aerosolized, which is in fluid contact with the upper end of the aerosol generator 301, and one or more key lock members complementary to the key lock members 104 of the base unit 100. A lid 304 closes the filling chamber 302 and prevents contamination or spillage of the liquid during use.

The base unit 100, the mouthpiece 200 and the aerosol head 300 are detachably connectible with one another. The device is assembled by inserting the mouthpiece 200 into the groove in the base unit 100, then placing the aerosol head 300 over the mouthpiece 200 and engaging the key lock member(s) 303 of the aerosol head 300 with the key lock member(s) of the base unit 100 by gentle pressure on both the aerosol head and the base unit. The aerosol generator 301 is positioned in the aerosol head 300 in such a way that when engaging the key lock member(s), the aerosol generator 301 is inserted into the lateral opening 202 of the mouthpiece 200. This creates airtight connections between the aerosol generator 301 and the lateral opening 202 in the mouthpiece as well as between the air outlet opening 102 of the base unit 100 and the air inlet opening 201 of the mouthpiece 200. The base unit 100, the mouthpiece 200 and the aerosol head 300 can be separated by reversing these steps.

The base unit 100 has one or more indentation(s) 106 positioned at or near the groove 103, and the mouthpiece 200 has one or more positioning member(s) 204. The indentation(s) of the base unit are complementary to (i.e. shaped to receive) the positioning member(s) of the mouthpiece. In this context, an indentation is a depression whose “negative” shape is complementary to the “positive” shape of a positioning member, such as a flange, projection or the like. Together, the indentations and positioning members act to position the mouthpiece correctly in the base unit. The indentation(s) and the positioning member(s) may be asymmetrical, so that the mouthpiece can only be inserted into the base unit in one way. This ensures that the device is assembled in such a manner that the position and orientation of the mouthpiece and base unit relative to each other are correct. The base unit contains a controller, such as a printed circuit board (PCB) which controls the operation of the nebulizer.

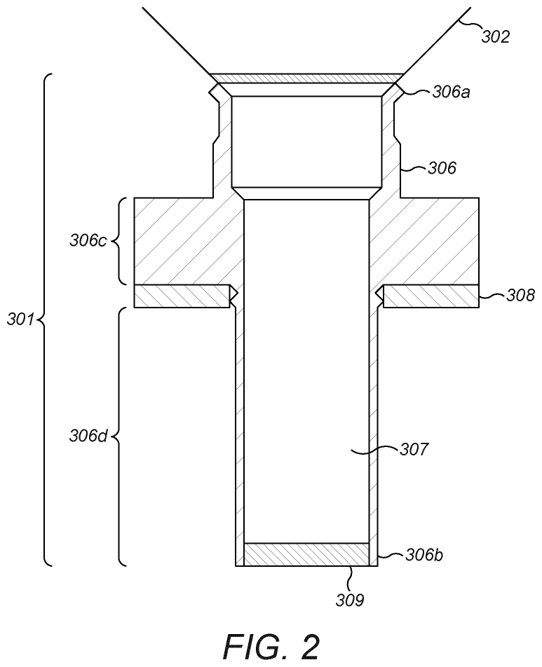

FIG. 2 shows the aerosol generator, which is described in detail in WO2008/058941. It comprises a vibrator, e.g. a piezoelectric element 308, a transducer body 306 and a membrane 309. The piezoelectric element is preferably an annular single or multi-layer ceramic, which vibrates the transducer body in a longitudinal mode. The transducer body is, for example made of stainless steel, titanium or aluminium, and encloses a cavity 307 which contains liquid to be aerosolized. The inside of the filling chamber 302 is conical so that liquid flows under gravity into the upstream end 306a of the transducer body and down into the cavity. Together, the filling chamber 302 and cavity 307 form a reservoir for the liquid.

The membrane 309 is positioned at the downstream end 306b of the transducer body 306. The holes in the membrane may be formed by electroforming or by laser drilling, with openings normally in the range from about 1 μm to about 10 μm. Without vibration of the membrane, the balance of pressures, the shape of the holes and the nature of the material used for the membrane are such that the liquid does not seep out through the membrane. However, vibration of the membrane leads to the formation and emission of aerosol droplets through the holes. The membrane may be made of plastic, silicon, ceramic or more preferably metal, and may be affixed onto or into the downstream end of the transducer body by various means, such as gluing, brazing, crimping or laser welding. Optionally, the membrane at least partially forms a dome in its central region, which causes the jet of nascent aerosol droplets to diverge and hence reduces the risk of droplet coalescence.

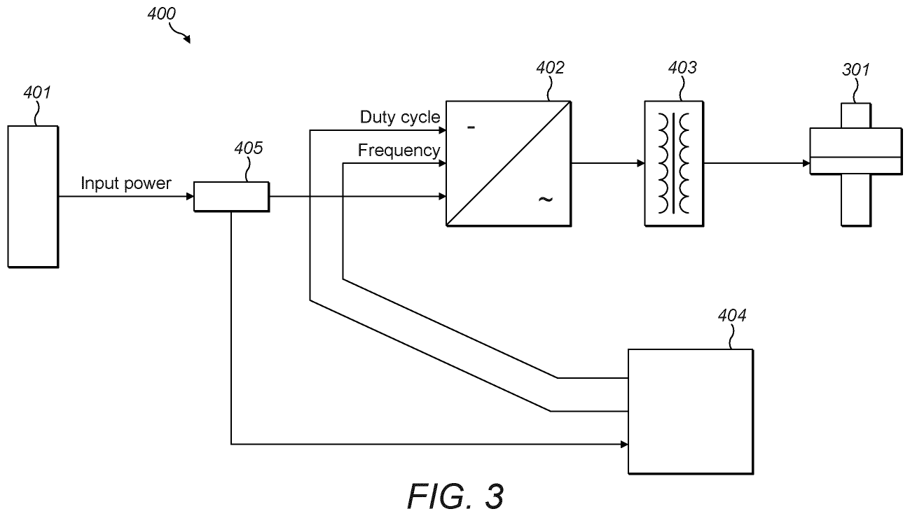

A driver circuit 400, shown schematically in FIG. 3, generates the driver signal that excites the piezoelectric element and hence causes the membrane to vibrate, typically at a frequency in the range of 50-200 kHz. The input dc power is provided by a battery 401. This is converted into an ac driving voltage by a power converter 402 and a transformer 403. A closed-loop controller 404 controls the power supplied to the aerosol generator 301 by pulse width modulation, by varying the duty cycle, i.e. the fraction of time for which the power is supplied to the aerosol generator. The controller 404 inputs the driving frequency and the duty cycle to the power converter 402. The controller 404 also measures the current consumed by the aerosol generator 301, by means of a shunt resistor 405 in series with the input of the power converter 402. The effective power consumption and the absolute value of the impedance can be derived from the measured current. The aerosol generator is driven using near-resonance driving in which the frequency of the driver signal is as a fixed offset (such as 500 Hz or 1 kHz) from the resonant frequency of the aerosol generator (typically around 85 kHz).

Excitation of the piezoelectric element causes micronic longitudinal displacements and/or deformations in a direction parallel to the symmetry axis of the transducer body 306. The transducer body has a region close to the piezoelectric element 308 with a relatively large wall thickness, which serves as a stress concentration zone 306c, and a region downstream thereof 306d with a relatively low wall thickness which serves as a deformation amplification zone. This configuration amplifies the vibrations or deformations of the transducer body 306 caused by the piezoelectric element 308. The piezoelectric element 308 is located at the level of, or adjacent to, the stress concentration zone 306c. The internal diameter of the transducer body at the deformation amplification zone may be the same as at the stress concentration zone, so that the differences in wall thickness correspond to different external diameters. Alternatively, the external diameter of the transducer body may be constant, while the inner diameters differ at the position of the two zones.

The nebulizer is breath-actuated so that it only generates aerosol when the patient is inhaling. This avoids wasting the aerosol that is generated when the patient is exhaling, as can occur in nebulizers that operate in a continuous manner. A pressure sensor (e.g. a barometric pressure sensor) is located adjacent to, and in pneumatic connection with, the channel in the base unit between the air inlet opening(s) and the air outlet opening 102. The pressure sensor measures the pressure in the channel, and sends a signal representing the pressure to the controller. When the patient begins to inhale on the mouthpiece, the pressure in the channel drops. If the pressure drops below a certain value, the controller determines that the patient has begun to inhale, and causes the piezoelectric element, and hence the membrane to vibrate, so that aerosol droplets are generated.

When the nebulizer is operated, the aerosol generated by the membrane 309 is released into the channel 205. Air enters through the air inlets in the base unit and passes through the channel in the base unit, the air outlet opening 102, and the air inlet opening 201 of the mouthpiece component, and into the channel 205 where mixes it with the aerosol. The air and aerosol then flow along the channel 205, out through the aerosol outlet opening 203 of the mouthpiece and into the patient's airway.

The controller stops the aerosol generation when a pre-set length of time (for example 3s) has elapsed since the aerosol generation started. The pre-set length of time may correspond to the length of a typical inhalation, and may be configurable by the patient. Alternatively, the pre-set length of time may be shorter than a typical inhalation, so that in the final part of the inhalation, the patient receives air but no aerosol. This ensures that the aerosol reaches the central and lower parts of the patient's airway, but is not delivered to the patient's upper airway (e.g. the throat) where it would be ineffective. However, the controller could alternatively detect when the patient ceases to inhale by sensing the increase in pressure in the channel, and then stop aerosol generation.

The resonant frequency of the aerosol generator changes over the duration of a treatment as the amount of liquid in the reservoir decreases. In order to maintain a fixed offset between the driver signal frequency and the resonant frequency, it is necessary to measure the resonant frequency at intervals throughout operation of the aerosol generator, for example every 0.5 s. This is done by scanning the frequency of the driver signal across a range of frequencies from below the resonant frequency to above it, for example from about 10 or 15 kHz below the resonant frequency to about 10 or 15 kHz above the resonant frequency, such as from 75 kHz to about 100 kHz in steps of 0.1 kHz. At each frequency, an electrical parameter which relates to the vibration of the aerosol generator is measured, for example the current consumption of the aerosol generator. The resulting graph of current as a function of frequency (the spectrum) has a peak at the resonant frequency of the aerosol generator. The scans take, for example, about 70 ms to perform. During the scans, the aerosol generator does not operate at the optimum frequency, so the aerosol output rate drops. Consequently, almost all of the aerosol is generated in the time between the scans (430 ms in this case).

The point at which the membrane becomes dry can be determined from the scans, for example from changes in the shape of the spectrum as a function of time, or in comparison to a standard spectrum, as described, for example in US2006/0102172, U.S. Pat. No. 9,272,101, WO2014/062175 and WO2015/091356. However, as discussed above, these methods may produce erroneous results as a result of variations in the hardware, changes in the hardware over the lifetime of the nebulizer and changes in external conditions.

The invention is based on a different effect which is independent of these variations, and so is more reliable. When the nebulizer is switched off, the liquid in the cavity has a flat surface with a meniscus around the edge. When the nebulizer is switched on and the aerosol generator is vibrated at or close to its resonant frequency, a standing wave is formed in the liquid within about 50 ms. The inverse effect is observed when vibration is stopped, although it takes longer (about 1 s) for the wave to dissipate and the liquid surface to become flat again.

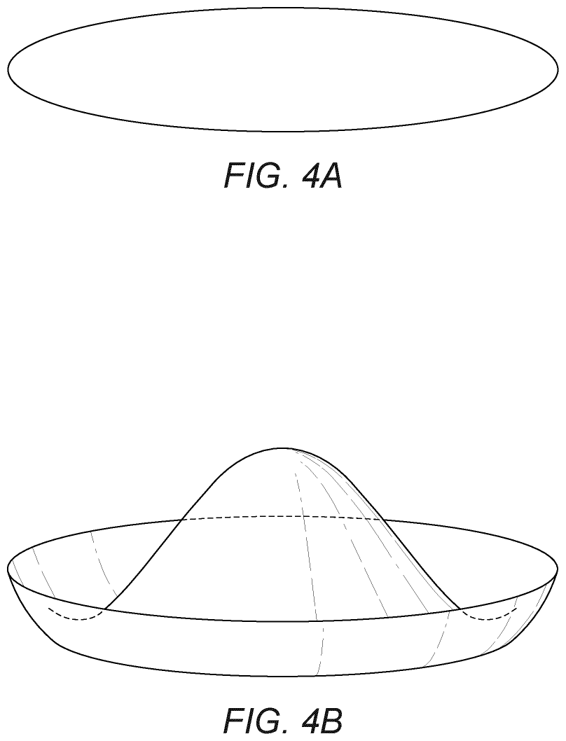

FIG. 4 shows schematic drawings of the surface of the liquid in the reservoir of a nebulizer of FIG. 1. FIG. 4A shows the liquid before vibration was started; the surface is flat, i.e. there is a uniform distribution of the liquid across the transducer body. FIG. 4B shows the surface during vibration; the liquid has formed a standing wave with a peak in the centre.

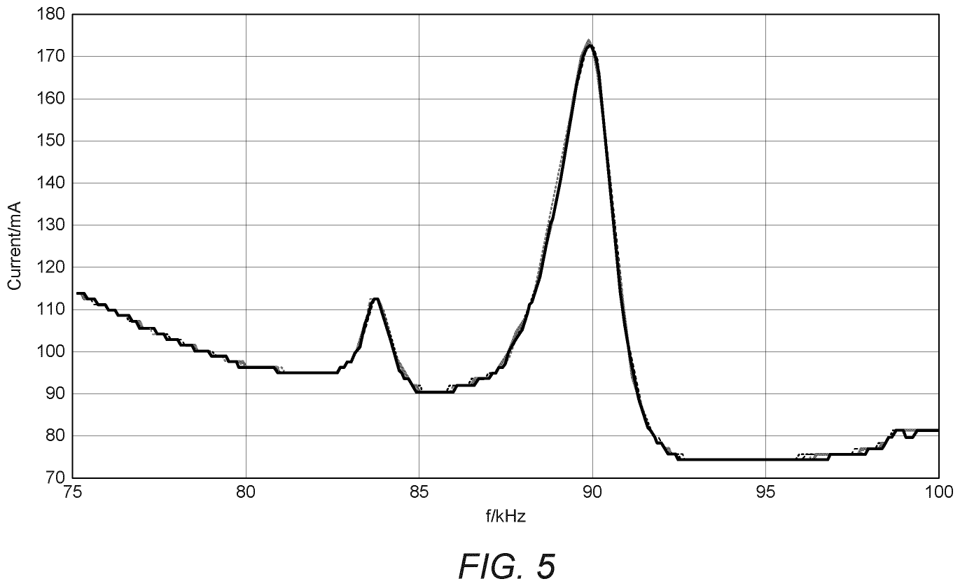

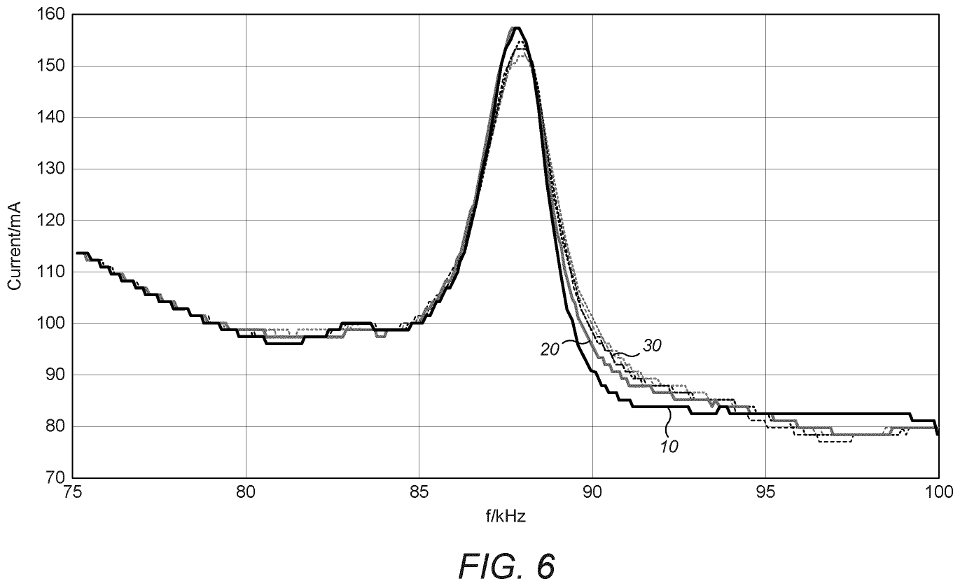

FIGS. 5 and 6 show spectra obtained with the nebulizer of FIG. 1, by vibrating the membrane at a series of different frequencies from 75 kHz to 100 kHz in steps of 0.1 khZ with a constant duty cycle, and measuring the current consumption of the power converter at each frequency. Seven scans, each taking 70 ms, were performed at 500 ms intervals. Between the scans, the membrane was vibrated at its normal near-resonant driving frequency for 430 ms.

FIG. 5 shows the resulting spectra when no liquid was present. The main peak at 89.5 kHz, at which the maximum current consumption occurs, is the resonant frequency of the aerosol generator. There is also a smaller peak at about 83 kHz, which is the resonant frequency of the membrane. No liquid is present so a standing wave is not formed; consequently, the electro-mechanical characteristics of the aerosol generator do not change between the scans. Thus, although each spectrum is plotted as a separate line, they almost exactly overlie each other, and it is not possible to distinguish between them.

FIG. 6 shows the spectra obtained when liquid was present. There are differences between the general shape of the spectra compared to those of FIG. 5. Firstly, the main resonance peak is at a lower frequency (86.5 kHz) and is slightly broader than in FIG. 5, i.e. the resonance is slightly less sharp when liquid is present. These changes in the main resonance peak over the course of a number of inhalations form the basis for some known methods of empty detection. Secondly, the smaller peak has disappeared.

Moreover, in contrast to FIG. 5, there are also clearly visible differences between the spectra in FIG. 6, due to the re-distribution of liquid as the standing wave is formed when the aerosol generator is switched on. The resonance peak moves to a slightly higher frequency, and becomes slightly broader in each successive spectrum. The largest difference is between the first spectrum 10 and the second spectrum 20. The third 30 and subsequent spectra are similar to each other, since the liquid had already been re-distributed when these were measured.

Since the these changes only occur when liquid is present, they can be used to distinguish between wet and dry states of the membrane. This forms the basis of the present invention. Thus, instead of comparing a measured value or spectrum with a pre-set value or spectrum, or comparing spectra obtained in different inhalations to identify changes that occur over the course of a treatment, the invention compares a spectrum obtained when aerosol is not being generated with a spectrum obtained after aerosol generation has begun. If no liquid is present, there is no standing wave so the spectra before and during vibration are the same. However, if liquid is present, the spectra are different: the first spectrum reflects the initial, flat liquid surface and the subsequent spectrum reflects the standing wave.

The first spectrum in FIG. 6 (at t=0 s, before aerosol generation) can be compared with one (or more) of the subsequent spectra, preferably the second spectrum (at t=0.5 s). The effect is most clearly seen between the first and second spectra because most of the redistribution of liquid occurs quickly, typically within about 50 ms, i.e. before the second spectrum is obtained. This difference is also present when comparing the first spectrum with any subsequent spectrum. However, the spectra are also affected by the liquid fill level in the reservoir. There is very little change in the fill level between the times at which the first and second spectra are recorded. In contrast, if the first spectrum is compared with, for example the seventh spectrum (t=3 s), the difference between them would reflect the decrease in the liquid fill level as well as the formation of the standing wave. Thus while the comparison could also be made using subsequent spectra, this is less preferred because these spectra reflect a combination of the two effects.

The degree to which two spectra match each other can be represented by an “overlap function”. The overlap function can be calculated as the reciprocal of the sum of the absolute value of the difference between the spectra at each frequency. Thus, when the spectra differ (e.g. the first and second spectra in FIG. 6), the sum of the absolute differences is large, and its reciprocal is small; hence the overlap function has a low value. On the other hand, when the spectra are very similar (as in FIG. 5) the overlap function has a higher value.

A representative treatment operation was performed using the nebulizer of FIG. 1 to nebulize 4 mL of 0.9% saline solution. The treatment operation consisted of 195 inhalations, i.e. about 20 μL of solution was nebulized in each inhalation. Aerosol generation was started when the patient's inhalation was detected and continued for 3 s in each inhalation. Seven scans were performed (at t=0, 0.5 s, 1 s, 1.5 s, 2 s, 2.5 s and 3 s) during each inhalation. Each scan took 70 ms. The resonant frequency was determined from the peak in each scan. Between the scans, the membrane was vibrated for 430 ms at a frequency 470 Hz above the resonant frequency, as determined from the immediately preceding scan.

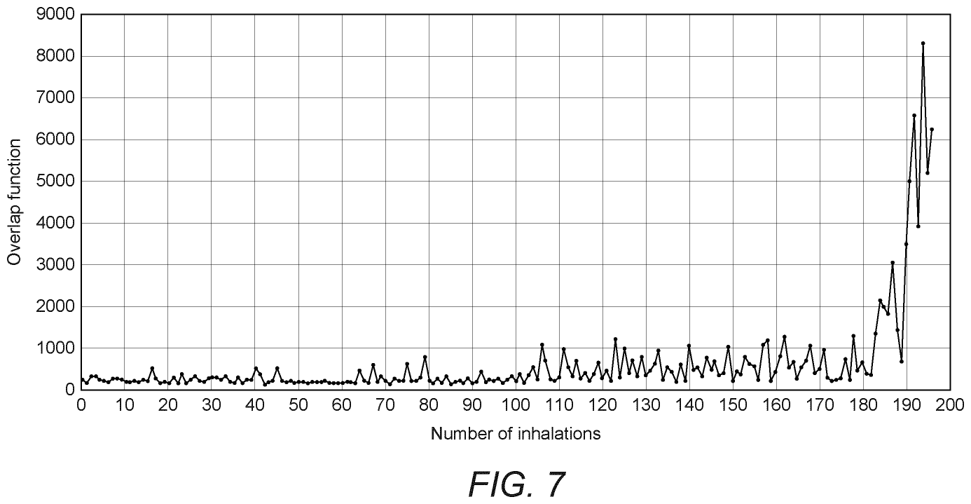

FIG. 7 shows a graph of the overlap function for each inhalation in the representative treatment operation. The overlap function is approximately constant at a low value (mostly below 1000) for the first 180 inhalations. This indicates that the first and second spectra differ in these inhalations, because a standing wave is formed in the liquid when the membrane starts to vibrate. Thereafter, the value of the overlap function rises sharply, indicating that the first and second spectra are becoming more similar in breaths 180-190, i.e. the standing wave effect is disappearing because there is very little liquid remaining.

A pre-set threshold can be used to determine the point at which the reservoir no longer contains liquid. A threshold value of e.g. 5000 would be suitable in FIG. 7. The determination of when the liquid has been used up can be made more robust by only deciding that the reservoir is empty if the value of the overlap function is greater than the threshold for a number of consecutive breaths (e.g. three or five breaths). This prevents incorrect determinations resulting from noise or an erroneous measurement in the overlap function.

Using the first and second spectra means that the overlap function can be calculated slightly sooner than if a subsequent spectrum were used, because the time delay between the first and second spectra (0.5 s) is smaller than, for example, between the first and seventh spectra (3 s). This has the advantage that the determination of when the threshold has been crossed can be made earlier (by 2.5 s in this example), so that the vibration of the membrane is stopped as soon as possible.

The invention is particularly suitable for breath-actuated nebulizers, since the vibrator is necessarily operated intermittently, i.e. only when the patient inhales. It could also be used in nebulizers that normally operate continuously, by introducing periods in which the vibrator is switched off. The duration of the off periods should be at least about 0.5 s, preferably 1 s, in order to allow the liquid to return to a flat surface before vibration is re-commenced.

The overlap function in FIG. 7 was obtained by comparing the spectra immediately before and during a period of aerosol generation, so that the difference between them reflects the formation of a standing wave when the membrane starts to vibrate. In other words, a spectrum obtained during a period of aerosol generation is compared with a spectrum obtained during the preceding period of little or no aerosol generation. However, the overlap function could equally be obtained by comparing a spectrum obtained during a period of aerosol generation with a spectrum obtained during the succeeding period of little or no aerosol generation. The difference between the spectra during and after aerosol generation reflects the dissipation of the standing wave when the membrane ceases to vibrate. This requires that sufficient time has elapsed after vibration ceases before the spectrum is measured. Typically the standing wave takes longer to dissipate (500-1000 ms) than it takes to form (about 50 ms). This is not a concern in a breath-actuated inhaler, since the time between inhalations is usually more than 1 s. However, in a nebulizer which is not breath-actuated, the periods in which the membrane is not vibrated would need to be longer to allow for dissipation, which would result in a reduction in the overall aerosol output rate.

The invention is especially suitable for nebulizers of the type shown in FIG. 1, in which regular scans are performed in order to determine the resonant frequency. The method of the invention can use the spectra from these scans, and extract additional information from them. Hence implementing the invention requires only some additional analysis of the spectra, and there is no need to change the way in which the nebulizer is operated.

The principle of the invention applies to any vibrating membrane nebulizer in which the membrane is in contact with a liquid reservoir in which a standing wave can be formed. Thus the invention can be used with other types of nebulizer, for example those described in WO2012/046220, WO2015/193432, WO2015/091356, US2006/0102172 and U.S. Pat. No. 9,027,548. These nebulizers do not have a transducer in the form of a hollow tubular body. Instead, the membrane is mounted directly on the piezoelectric element, or there is an annular, planar support member on which the membrane and/or the piezoelectric element are mounted.

The method of the invention could be used instead of, or in addition to other empty detection methods (for example as in US2006/0102172, U.S. Pat. No. 9,272,101, WO2014/062175 and WO2015/091356 which measure the changes in an electrical parameter as the volume of liquid decreases over time) to provide a combined decision process for determining whether membrane is dry. Since the method of the invention relies on a completely different effect from these other methods, it provides completely independent information on whether liquid is present. Thus the combination of the method of the invention and a different method provides particularly robust empty detection.

1. A breath-actuated inhalation device comprising an aerosol generator having a vibrator and a membrane, a reservoir for liquid to be aerosolized which is in fluid communication with the membrane, and a controller that provides a driver signal to drive the vibrator so that the membrane vibrates and generates an aerosol, wherein the controller is configured to:
drive the vibrator intermittently so that the aerosol generator repeatedly undergoes periods of aerosol generation during a patient's inhalations and periods of little or no aerosol generation preceding and/or succeeding the inhalations;
perform scans in which the membrane is vibrated at a plurality of frequencies, and in which at least one electrical parameter of the vibrator is measured at the plurality of frequencies to provide a spectrum; wherein the scans are performed during the inhalations and during the periods preceding or succeeding the inhalations;
compare a spectrum obtained during an inhalation with a spectrum obtained during the period preceding or succeeding that inhalation;
determine whether liquid is present in the reservoir on the basis of the comparison of the spectra; and
cease to drive the vibrator if the controller determines that no liquid is present.

2. The inhalation device according to claim 1 wherein the controller is configured to perform a first scan before each inhalation to obtain a first spectrum, to subsequently perform a second scan during each inhalation to obtain a second spectrum, and to compare the first and second spectra.

3. The inhalation device according to claim 1, further comprising a channel having an air inlet opening and an aerosol outlet opening, and a pressure sensor which is pneumatically connected to the channel, wherein the controller is configured to detect inhalation by a patient at the aerosol outlet opening on the basis of a signal from the pressure sensor, and to initiate a period of aerosol generation in response to the inhalation.

4. The inhalation device according to claim 3, wherein the controller is configured to initiate a period of little or no aerosol generation at a pre-set time after the period of aerosol generation was initiated.

5. The inhalation device according to claim 1, wherein the aerosol generator further comprises a support member comprising a hollow tubular body having a flange at or close to a first end onto which the vibrator is attached, and a second end into or onto which the membrane is mounted, and wherein the device comprises a filling chamber above the support member, so that the filling chamber and the hollow tubular body together form the reservoir.

6. The inhalation device according to claim 1, wherein the controller is configured to determine the resonant frequency of the aerosol generator from the spectra, and to drive the vibrator at the resonant frequency, or at a frequency related to the resonant frequency, during the periods of aerosol generation other than the scans.

7. The inhalation device according to claim 6, wherein the plurality of frequencies comprises from about 10 or 15 kHz below the resonant frequency to about 10 or 15 kHz above the resonant frequency, for example from 75 kHz to about 100 kHz.

8. The inhalation device according to claim 1, wherein the controller is configured to compare the spectra by calculating an overlap function.

9. The inhalation device according to claim 8, wherein the controller is configured to determine that no liquid is present in the reservoir if the overlap function is above a threshold value.

10. The inhalation device according to claim 9, wherein the controller is configured to cease driving the vibrator if the overlap function is above the threshold value for a plurality of consecutive periods of aerosol generation, such as three or five periods.

11. A method of operating a breath-actuated inhalation device comprising an aerosol generator having a vibrator and a membrane, and a reservoir for liquid to be aerosolized which is in fluid communication with the membrane, the method comprising:
a) driving the vibrator intermittently so that the aerosol generator repeatedly undergoes periods of aerosol generation during a patient's inhalations and periods of little or no aerosol generation preceding and/or succeeding the inhalations;
b) performing scans in which the membrane is vibrated at a plurality of frequencies, and in which at least one electrical parameter of the vibrator is measured at the plurality of frequencies to provide a spectrum; wherein the scans are performed during the inhalations and during periods of little or no aerosol generation preceding and/or succeeding the inhalations;
c) comparing a spectrum obtained during an inhalation with a spectrum obtained during the period preceding or succeeding that inhalation;
d) determining whether liquid is present in the reservoir on the basis of the comparison of the spectra; and
e) ceasing to drive the vibrator if it is determined in step d) that no liquid is present.

12. The method according to claim 11 wherein a first scan is performed before each inhalation to obtain a first spectrum, a second scan is subsequently performed during each inhalation to obtain a second spectrum, and the first and second spectra are compared.

13. The method according to claim 11, wherein the inhalation device comprises a channel having an air inlet opening and an aerosol outlet opening and a pressure sensor which is pneumatically connected to the channel, wherein the periods of aerosol generation are initiated in response to inhalation by the patient on the basis of a signal from the pressure sensor.

14. The method according to claim 11, wherein in step c), the spectra are compared by calculating an overlap function.

15. The method according to claim 14, wherein in step d), it is determined that no liquid is present in the reservoir if the overlap function is above a threshold value, and preferably wherein in step e), driving the vibrator ceases if the overlap function is above the threshold value for a plurality of consecutive inhalation, such as three or five inhalations.
